(19) United States
(12) Patent Application Publication
(10) Pub. No.: US 2022/0237709 A1
(43) Pub. Date: Jul. 28, 2022

(54) Sensor Tracking Based Patient Social Content System

(71) Applicant: Happify, Inc., New York, NY (US)

(72) Inventors: Ran ZILCA, Ra'anana (IL); Keren ZIMMERMAN, New York, NY (US)

(21) Appl. No.: 17/585,003

(22) Filed: Jan. 26, 2022

Related U.S. Application Data
(60) Provisional application No. 63/141,538, filed on Jan. 26, 2021.

(51) Int. Cl. G06Q 50/00; G16H 40/67

(52) U.S. Cl. CPC G06Q 50/01; G16H 40/67

(57) ABSTRACT

A computing system that includes a server, the system functions as a patient social content system (PSCS) which, among other functions, provides content to a patient/user. A device with a network connection to the computing system permits the patient to provide information to the computing system. In addition, one or more sensors are configured to detect changes in various physical parameters relevant to the patient. Electrical signals from the sensors are conveyed to the computing system so that the computing system is aware of changes in physical parameters of the patient. Based on the changed physical parameters of the patient, the computing system provides different content to the user that is relevant to the changes. Overall, processes carried out by the system establish and maintain a patient social content system that utilizes tracking data received from one or more sensors.

### CROSS-REFERENCE TO RELATED APPLICATIONS

This application claims the benefit of U.S. Provisional Application No. 63/141,538, filed on Jan. 26, 2021. The entire disclosure of the above application is incorporated herein by reference.

### FIELD OF INVENTION

The present invention is directed to a computing system, and a process carried out by such system, for establishing and maintaining a patient social content system that utilizes tracking data received from one or more sensors.

### BACKGROUND

A patient support group is a group of people with common diagnoses, experiences and concerns regarding a disease or several related diseases. The members of the group provide emotional and moral support for one another. Patient support groups serve a number of positive functions which include educating patients themselves, their family members, sharing experience regarding common diagnoses, sharing information regarding related diagnoses, supporting its members, exposing patients to treatment options and raising public awareness.

A wide variety of support groups have been available for patients with similar disease diagnoses throughout history. Use of social media and the Internet has greatly increased the reach and applicability of these groups. Surprisingly, even with the available tools allowing for nearly effortless participation in such groups, engagement in patient support groups remains surprisingly low, with some estimates putting participation in the 10% range for serious diagnoses such as head and neck oncology. Reflections: The Value of Patient Support Groups, Otolarynol Head Neck Surg, 156 (4), pp. 587-588 (2017). One potential reason for this low participation rate is an excess of information. That is, even with groups highly focused on a particular disease, a participant may be overloaded with information irrelevant to their particularized needs. As such, the ability to provide a patient with information highly relevant to their particular needs while screening-out less relevant information has the potential to increase support group participation rates.

A patient social content system or “PSCS” is a system that allows patients to create an account specifying personal information regarding themselves, information regarding their medical history, the type of information they seek, what information they may be able to provide to other patients, their privacy settings, etc. One key element of personal information on a PSCS is any diagnosis or diagnoses pertinent to the patient.

Once an account is created and sufficient information regarding the patient, e.g., identifying information and diagnoses, information flows to the patient and from the patient via the PSCS. The various content shown to patients in a PSCS may be presented in a “feed” such that more relevant content, both from other patients and editorial, should be shown at the top of the feed. This sorting by relevance can be done in various ways and based on various types of input. Feeds may be provided generally to all patients on a PSCS or may be more personalized, depending upon patient information such as diagnoses, stage of a disease affecting the patient, patient preferences and level of interaction with previous content provided to the patient. The presentation of content to a patient by the PSCS should depend greatly upon the relevance of that information to the patient. How interesting and useful other patients found certain content may also be part of this relevance determination.

The patient is able to browse through posts/content contributed by other patients as well as articles and similar content contributed editorially by the team that manages the PSCS. The patient will engage with PSCS content by, for example, liking it, marking it as helpful, commenting on it, or commenting on comments. The patient may also explicitly draw the attention of certain other patients to the content, e.g., by sending the content to them.

Alternatively, a patient may draw the attention to content by making it part of their PSCS profile and making portions of their PSCS profile viewable to other patients.

### OBJECTS AND SUMMARY OF THE INVENTION

In view of the foregoing, it is an object of the present invention to provide a computing system/method for operating a patient social contact system (“PSCS”) that receives sensor data from sensors ‘external’ to the PSCS. It is another object of the present invention to utilize additional sensor data to improve the operation of the PSCS, especially regarding the provision of relevant content to patients. That is, content is provided to a particular patient by the PSCS that is highly relevant to that patient and that takes into account as much data as possible pertinent to the circumstances of that patient.

In accordance with an embodiment of the present invention, a computing system for interacting with a patient is provided, in which the computing system commences, with a patient, a PSCS designed to cause an increase in physical and emotional well-being of the patient. The PSCS, among other things, presents content to the patient relevant to one or more disease conditions from which the patient is suffering. These conditions align with one or more diagnoses that may have been given to the patient by medical professionals or merely of interest to the patient or other user, e.g., a care-giver.

Information, i.e., data, input to the PSCS from the patient forms at least a portion of the received input data through which the PSCS may determine content to be made available to the patient. Said data may include text responses from the patient to which semantic analysis may be applied to identify terms related, for example, to particular diagnoses or disease states. The patient text responses may be the result of the computing system generating prompts designed to elicit, from the patient, a response. The prompts and patient responses may be in the form of text. In addition, either or both of the prompts and responses may be in the form of an audible speech signal using text-to-speech and speech-to-text subroutines, as applicable. User behavior in the form of click stream, interaction paths and preferences may also form part of the data input to the PSCS. Pictures/video may also form the basis of data input to the PSCS. Extraction or synthesis of a textual description based on images/video may be the basis for this data.

In addition to the data provided by the user within the PSCS, another source of data input to the PSCS may include information from sensors not typically associated with a PSCS. For example, data from the following sensors may be presented to the computing system operating the PSCS: cameras, microphones, heat sensors motion sensors, fingerprint detectors, keyboards, accelerometers or other motion detectors, GPS or other position sensors, activity sensors, pedometers, biochemical sensors, medical sensors, pressure sensors, brainwave sensors, sleep cycle sensors, heart rate sensors, facial expression sensors, voice tone sensors and image sensors. Medical sensors include any device that senses a physiological condition or change in the patient, e.g., heart rate, ECG, respiration, blood glucose, oximetry, etc. A sensor integrated with a device such as a smartphone, watch, laptop, larger medical device, network appliance or similar electronic device may be considered a sensor in accordance with the present description to the extent in provides data directly or indirectly to the PSCS. Whether a sensor is integrated with a device already in use by the user is not indicative of whether or not it is “typically associated with a PSCS”, many such sensors are not typically associated with a PSCS.

In accordance with another embodiment of the present invention, a method of interacting with a patient by a computing system is provided, in which the inventive method comprises a PSCS capable of receiving data from sensors associated with the patient but external to the PSCS system. The method includes processing of data from the external sensors such that the functions of the PSCS are performed more effectively. In particular, the method increases the ability of the PSCS to make determinations regarding the relevance of content to the patient. These and other objects, advantages, aspects and features of the present invention are as described below and/or appreciated and well understood by those of ordinary skill in the art. Although specific advantages have been enumerated above, various embodiments may include some, none, or all of the enumerated advantages and other technical advantages may become readily apparent to one of ordinary skill in the art after review of the following figures and description.

### BRIEF DESCRIPTION OF THE FIGURES

FIG. 1 is a block diagram of an exemplary computing system in accordance with the present invention.

FIG. 2 is an exemplary flow chart showing an overview of steps carried out by an exemplary embodiment of the present invention.

### DETAILED DESCRIPTION

The present invention is directed to a patient social content system (“PSCS”) operating on a computing system to which external sensors provide data. Although exemplary embodiments are presented in the figures and described below, the present disclosure may be implemented using any number of techniques, whether currently known or not. The present disclosure should in no way be limited to the exemplary implementations and techniques illustrated in the figures and described below.

The term “patient social content system” or “PSCS” as used herein is intended to be construed broadly, and as such, the term may include a variety of systems designed specifically to generate and disseminate content relevant to a group of patients with similar or related diagnoses. Content may be generated for the PSCS by the patients themselves or by an administrator of the PSCS. An important aspect of any PSCS is that content is provided to a particular user, here a particular patient, based on data related to that patient collected by the PSCS.

In accordance with the present invention, the term “data” should be construed broadly to encompass any information provided to the computing system that is relevant to a patient or group of patients.

The term “sensor” is a device that is able to receive a physical stimulus such as heat, light, sound, pressure, magnetism, or a particular motion and transmits an impulse responsive to the stimulus. An electronic sensor detects events or changes in its environment and sends an electronic signal to other electronic components, e.g., to a computer processor.

The term “content” refers to posts, blogs, articles, guides, ebooks, links (to internal/external content), images, videos, video stories, live videos, infographics, testimonials, reviews, announcements, contests, promotions, tools, and advertisements, among other things that may be incorporated as part of a social media feed.

A patient joining a PSCS begins by creating an account and providing general information regarding themselves, e.g., demographic information, as well as information specific to their health and any conditions from which they are suffering or for which they are at risk. A patient's current condition status, including any diagnosis or diagnoses relevant to the patient and treatment(s) currently being utilized, forms an important set of data points for the PSCS. Among many other classes of data provided to the system, the patient may also provide information specifying which information they seek, which information they can provide to other patients, their privacy settings, etc.

After collecting sufficient information regarding the patient, the PSCS is able to provide content to the patient. Also of importance, the PSCS remains open to receiving or calculating additional data regarding the patient. Information may be provided to the patient in a number of ways. One way content may be presented to a patient is in the form of a feed. The feed may be purely chronological, in a form stressing the relevance the PSCS places on particular content for a patient, some other order or a combination of these options. In an embodiment, content from other patients or the PSCS editors should be ordered on a patient's feed based on the relevance the PSCS system attributes to that content and how recently that content was posted on the PSCS. The presentation of content to a patient by the PSCS should, to one degree or another, be related to the relevance of that content to the patient.

Sorting content to be provided to patients, e.g., on their feed, by relevance can be done in various ways and based on various information/data. The PSCS system and computer processor integrated therewith has the ability to process all data pertinent to a given patient and, based on this data, attach relevance scores to content vis-à-vis that particular patient. The data pertinent to a given patient is sourced from information provided to the PSCS by the patient themselves, information from third party sources and information provided to the PSCS by sensors described with more particularity hereinbelow.

A relevance score attached to content may be based upon, for example, patient information such as diagnoses and stage of a disease affecting the patient. Relevance scores may also be adjusted iteratively based on how interesting and useful other patients found certain content and how similar or dissimilar these other patients are to the patient for whom a relevance score is being calculated. Shared diagnoses may be an important factor in making these iterative relevance score calculations.

Content added to a patient's feed may include information contributed editorially by the team that manages the PSCS or contributed by other patients.

Patients may engage with content by marking it as interesting to them, marking it as helpful, commenting on it or commenting on comments. A patient may also send content to other patients or to persons outside of the PSCS. A patient may also draw attention to content by making it part of their PSCS profile and making portions of their PSCS profile viewable to other patients.

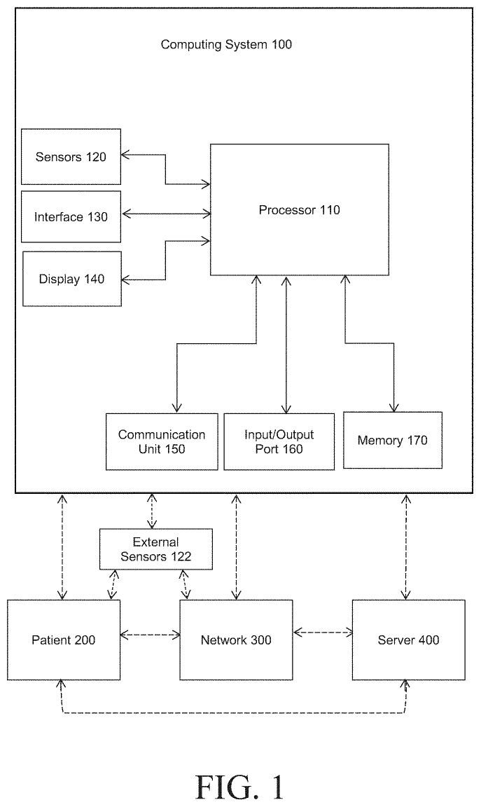

Referring now to the drawings in which like numerals represent the same or similar elements, and initially to FIG. 1 thereof, a computing system 100 configured in accordance with the present invention is illustratively shown in accordance with one embodiment. The computing system 100 includes one or more processors 110 that processes various input data and stored data and controls operations of other components within the computing system 100 to enable the herein described PSCS system and method. As will be further described, the processor 110 processes data by performing numerous mathematical algorithms and analytical computations. The processor 110 may also be a plurality of processing units that each carries out respective mathematical algorithm and/or analytical computation. In some embodiments, the processor 110 is enhanced by artificial intelligence.

The computing system 100 further includes a plurality of sensors 120. The plurality of sensors 120 may comprise a speaker/microphone, a still image camera, a moving image camera, a biometric sensor, etc. Each of the sensors 120 is configured to obtain patient input data and may further comprise one or more respective processing units to process the obtained input data in conjunction with the processor 110. The computing system 100 further includes an interface 130 to allow the patient 200 to operate the computing system and a display 140 to present information to the patient 200. In some embodiments, the interface 130 and the display 140 may come as one unit such as a touch screen display.

The computing system 100 further includes a communication unit/device 150, an input/output port 160 and a memory 170. The communication unit/device 150 allows the computing system 100 to communicate with the patient's other electronic devices or with additional sensors within a vicinity of the patient 200 over a network 300. The network 300 may include wireless communications, wired communications, etc. The network 300 may include the Internet, a wide area or local area network, etc. The computing system 100 may use the I/O port 160 for inputting and outputting data. The computing system 100 further includes the memory 170 which stores programs and applications. The memory 170 may store a database of content or may locally store content retrieved from a server 400 having thereon a database of content.

The computing device 100, as well as the patient's other electronic devices or the additional sensors, may be part of or otherwise be connected to the network 300 and coupled to a server or a service provider 400. The broken lines in FIG. 1 signify that the patient 200, the network 300, the server 400, the external sensors 122 and the computing system 100 may be connected to any one or more of the patient 200, the network 300, the server 400, the external sensors 122 or the computing system 100, either directly, indirectly, or remotely over a communication path. One or more of the computing system 100, the network 300, the external sensors 122 and the server 400 may be located on one computer, distributed over multiple computers, or be partly or wholly Internet-based.

The current invention aims to improve the PSCS format by increasing the relevance of content fed to the patient. Said improvement in content relevance is based on the information, i.e., data, collected by the PSCS regarding the particular patient. Based on this computed relevance, the PSCS selects content to send to the particular patient, which content is more likely to be of interest to the patient and interacted with by the patient. Relevant content is selected taking into account not only the data collected within PSCS sessions directly from the patient but also data collected in between sessions, from sources outside of the PSCS such as sensors as tracking devices.

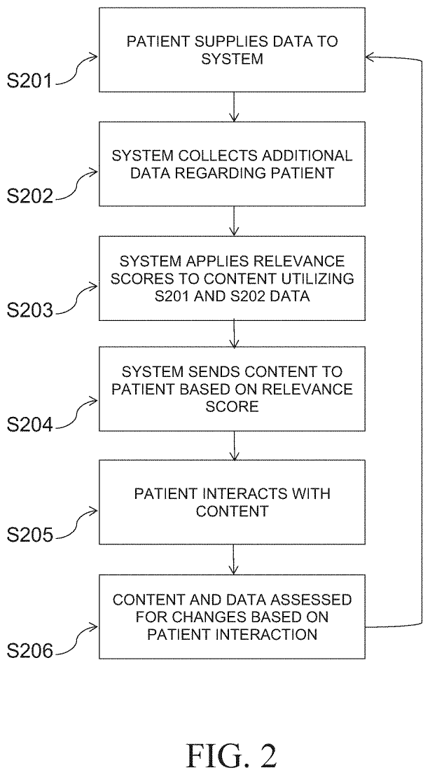

An overview of the steps carried out by an exemplary computing system in accordance with the present invention is shown in FIG. 2.

Step S201 involves the patient supplying certain information/data to the PSCS. This information may range from fairly basic to highly detailed information and should include at least some information as to medical history and/or diagnoses of the patient. Step S201 may involve the PSCS interacting with a patient in an iterative way (i.e., engaging in a conversation either via text or via voice). For example, an iterative interaction initiated by the computing system may comprise providing a user with a prompt, receiving input data from the user, providing a follow-up prompt to the user, receiving further input data from the user, etc. Step S201 need not precede the remaining steps, especially once a certain amount of information regarding the patient is already stored by the PSCS, as will be further discussed below.

Step S202 entails collecting data from sensors. These sensors may be integrated with the PSCS, i.e., sensors 120, or may be external sensors 122 remote from the PSCS. For example, the PSCS may have integrated sensors 120 or be in wired or wireless communication with external sensors 122 configured to collect user information. Sensors 120 and external sensors 122 include such things as cameras, microphones, heat sensors motion sensors, fingerprint detectors, keyboards, accelerometers or other motion detectors, GPS or other position sensors, activity sensors, biochemical sensors, medical sensors, pressure sensors, brainwave sensors, sleep cycle sensors, heart rate sensors, facial expression sensors, voice tone sensors and image sensors. Medical sensors include any device that senses a physiological condition or change in the patient. According to an embodiment, a sensor integrated with a device such as a smartphone, watch, laptop, larger medical device, network appliance or similar electronic device may be considered an external sensor 122 in accordance with the present description to the extent in provides data directly or indirectly to the PSCS.

In accordance with an embodiment, the data collected by sensors associated with the patient need not be limited to times when the patient is directly interacting with the PSCS. That is, sensors 120, 122 may also send data to the PSCS between sessions, i.e., when the patient is not logged into the PSCS. Since data outside the PSCS may be highly useful in achieving the functions of the PSCS, incorporation of such data may be highly useful to improving the utility of the PSCS to a patient.

The data collected by step S202 may include facts, conditions, location, movement, psychology, circumstances, physiological status or similar information regarding the patient. For example, data may be received or derived from one or more sensors associated with the patient. GPS, IP address, mobile tower, geofencing, other location data, etc., may reveal the user has not left their house for several days or, conversely, has had several days of non-stop activity, whether potentially indicative of a pathology or merely reflecting a hyperkinetic, not pathological, period. Sensors in the form of sleep monitors may also be used as external sensors 122. In addition, mobile devices such as smart phones may function as sleep monitors, being a proxy for wakefulness. That is, actual interaction with the mobile device by the user means that the user is not asleep. Physiological monitors, e.g., smart watch, Fitbit, blood pressure/heart rate monitor, glucose trackers, may also be utilized as sensors 120, 122. Many physiological monitors are also de facto sleep monitors. Sensors also encompass more traditional inputs like text and voice-to-text input from the user.

The potential exists for S201 and S202 to overlap in a number of areas as well as for one to occur while the other does not occur. Either way, the end product of S201 and/or S202 is the data used as an input to S203. Step S203 entails the assessment of data received from S201 and S202. This data may also be supplemented by data relevant to the patient saved in memory 170, e.g., data from previous interactions between the processor 110 and the patient. Memory 170 also contains or has access to a database full of content of varying levels of relevance to the patient; this ‘content database’ is constantly being updated with additional content from other patients and the PSCS editors.

Assessment of the data in step S203 may include calculation of relevance scores for individual elements contained in the content database. That is, the data collected and saved in steps S201 and S202 regarding a particular patient comprises an input to an algorithm which calculates a relevance score for elements stored in the content database in memory 170. All or some of the data relevant to the patient may be used by processor 110 in step S203. Further, step S203 may be applied to the entire content database or portions thereof, e.g., the ‘new’ content added to the content database over some predetermined time period.

In step S204 the PSCS sends or otherwise makes the patient aware of content most likely to be of interest to the patient. In accordance with an embodiment of the invention, the PSCS utilizes the relevance scores calculated in step S203 to make the determination as to what content to send to the patient. Thus, all information/data pertinent to the patient, collected in steps S201 and S202 and stored in memory 170 is available for increasing the likelihood that content sent to the patient is highly relevant to all of the patient's present circumstances and, thus, the likelihood that the patient will interact with the content in step S205.

The patient's interaction or lack of interaction with content in step S205 is also assessed by the PSCS. That is, in step S206 the PSCS assesses the details regarding interaction between the patient and an element of content. This interaction has the potential to impact both the data/information pertinent to the patient as well as the content stored in memory 170. The data pertinent to a particular patient may be modified by an interaction with content such that the algorithm as S203 is optimized to increase or decrease the relevance score of similar content. Additionally, the content interacted with may have its content database record adjusted such that future relevance scores calculated for it are increased or decreased. For example, a substantial interaction between a particular patient and a particular element of content may result in step S206 making a change to the content database record for that element of content such that its relevance score to other patients in similar circumstances will increase. The change to the content database record will result in the algorithm at step S203 calculating a higher relevance score for patients in similar circumstances to the particular patient that apparently found the content element highly relevant.

From step S206, the PSCS may return to collecting and assessing data at step S201 or S202; alternatively, the PSCS may return to S203. Thus, additional data may be collected regarding the patient prior to additional content being suggested to the patient or not.

It should be appreciated that data collected for use with/by the PSCS does not necessarily require interaction of the user with a device. Rather, some sensors may operate in the background continuously and not only when the user is using the device or the PSCS app. The user may not interact with the PSCS at all, yet receive different PSCS feeds and recommendations because sensors measured changes in their sleep, activity, blood glucose, etc., patterns. This is particularly true when it comes to sleep patterns, which may be determined or inferred based on a number of, to one extent or another, passive data streams from sensors associated with the user.

Further to the foregoing, the appreciation of established patterns and deviation from said patterns may comprise important data regarding the user. Such data may include triggers for the PSCS relevance score algorithm. Also, statistical inferences from such patterns may be used to extract higher level insights from the raw sensor data. A sleep quality estimate, which gains precision based on the volume of pattern data collected, is an example of such an inference. More directly connected to a disease state that may form the basis for the PSCS, pattern data may detect a flare-up/relapse in disease symptoms.

Appearances of the phrase “in an embodiment” or “in an exemplary embodiment,” or any other variations of this phrase, appearing in various places throughout the specification are not necessarily all referring to the same embodiment, and only mean that a particular characteristic, feature, structure, and so forth described in connection with the embodiment described is included in at least one embodiment.

The technology described herein may be incorporated in a system, a method, and/or a computer program product, the product including a non-transitory computer readable storage medium having program instructions that are readable by a computer, causing aspects of one or more embodiments to be carried out by a processor. The program instructions are readable by a computer and can be downloaded to a computing/processing device or devices from a computer readable storage medium or to an external computer or external storage device via a network, which can comprise a local or wide area network, a wireless network, or the Internet. Additionally, the network may comprise wireless transmission, routers, firewalls, switches, copper transmission cables, optical transmission fibers, edge servers, and/or gateway computers. Within the respective computing/processing device, a network adapter card or network interface in each computing/processing device receives computer readable program instructions from the network and forwards the computer readable program instructions for storage in a computer readable storage medium.

As used herein, a computer readable storage medium is not to be construed as being transitory signals, such as radio waves or other freely propagating electromagnetic waves, electromagnetic waves propagating through a waveguide or other transmission media, or electrical signals transmitted through a wire. The computer readable storage medium may be, but is not limited to, e.g., a magnetic storage device, an electronic storage device, an optical storage device, a semiconductor storage device, an electromagnetic storage device, or any suitable combination of the foregoing, and can be a tangible device that can retain and store instructions for use by an instruction execution device. The following is a list of more specific examples of the computer readable storage medium, but is not exhaustive: punch-cards, raised structures in a groove, or other mechanically encoded device having instructions recorded thereon, an erasable programmable read-only memory, a static random access memory, a portable compact disc read-only memory, a digital versatile disk, a portable computer diskette, a hard disk, a random access memory, a read-only memory, flash memory, a memory stick, a floppy disk, and any suitable combination of the foregoing.

The operations of one or more embodiments described herein may be carried out by program instructions which may be machine instructions, machine dependent instructions, microcode, assembler instructions, instruction-set-architecture instructions, firmware instructions, state-setting data, or either source code or object code written in any combination of one or more programming languages, including an object oriented programming language such as, but not limited to, C++, and other conventional procedural programming languages.

The program instructions, as will be clear to those skilled in the art from the context of the description, may have the capability of being executed entirely on a computer of a patient, may also be executed partly on the computer of the patient, partly on a remote computer and partly on the computer of the patient, entirely on the remote computer or server, or as a stand-alone software package. In the “entirely on the remote computer or server” scenario, the remote computer may be connected to the patient's computer through any type of network, including a wide area network or a local area network, or the connection may be made to an external computer. In some embodiments, electronic circuitry including, e.g., field-programmable gate arrays, programmable logic circuitry, or programmable logic arrays may execute the program instructions by utilizing state information of the program instructions to personalize the electronic circuitry, in order to perform aspects of one or more of the embodiments described herein. These program instructions may be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. These program instructions may also be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The computer readable program instructions may also be loaded onto a computer, other programming apparatus, or other device to produce a computer implemented process, such that the instructions which execute on the computer, other programmable apparatus, or other device implement the functions/acts specified in the flowchart and/or block diagram block or blocks.

The block and/or other diagrams and/or flowchart illustrations in the Figures are illustrative of the functionality, architecture, and operation of possible implementations of systems, methods, and computer program products according to the present invention's various embodiments. In this regard, each block in the block and/or other diagrams and/or flowchart illustrations may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently or sometimes in reverse order, depending upon the functionality involved. It will also be noted that each block of the block and/or other diagram and/or flowchart illustration, and combinations of blocks in the block and/or other diagram and/or flowchart illustration, can be implemented by special purpose hardware-based systems that perform the specified functions or acts or carry out combinations of special purpose hardware and computer instructions.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. To aid the Patent Office and any readers of any patent issued on this application in interpreting the claims appended hereto, applicant wish to note that applicant does not intend any of the appended claims or claim elements to invoke 35 U.S.C. § 112(f) unless the words “means for” or “step for” are explicitly used in the particular claim.

What is claimed is:

1. A sensor social content system comprising:
a. a computing system that includes a server and a plurality of networked devices, the plurality of networked devices including a patient device, the patient device permits a patient to interact with the computing system and the computing system to interact with the patient;
b. at least one sensor configured to report an electronic signal generated by the sensor to the computing system, wherein the electronic signal reports a change in the sensor environment which the sensor is adapted to detect; and
c. the computing system configured to provide content to the patient, the content determined based on patient interaction with the computing system and one or more changes in the sensor environment reported to the computing system by the electronic signal from the sensor.

2. The sensor social content system of claim 1 wherein content is selected from the group consisting of posts, blogs, articles, guides, ebooks, links to internal or external content, images, videos, video stories, live videos, infographics, testimonials, reviews, announcements, contests, promotions, tools and advertisements.

3. The sensor social content system of claim 1 wherein the at least one sensor is selected from the group consisting of camera, microphone, heat sensor, motion sensor, fingerprint detector, accelerometer, motion detector, GPS, position sensor, activity sensor, pedometers, biochemical sensor, medical sensor, pressure sensor, brainwave sensor, sleep cycle sensor, heart rate sensor, facial expression sensor, voice tone sensor, heart rate sensor, ECG sensor, respiration sensor, blood glucose sensor, oximetry sensor, medical sensor and image sensor.

4. A sensor social content system comprising:
a. a computing system that includes a server and a plurality of networked devices, the plurality of networked devices including a patient device, the patient device permits a patient to interact with the computing system and the computing system to interact with the patient;
b. the computing system configured to provide content to the patient in the form of a feed;
c. at least one sensor configured to report an electronic signal generated by the sensor to the computing system, wherein the electronic signal reports a change in the sensor environment which the sensor is adapted to detect; and
d. the feed is provided to the patient in a form that is sorted and prioritized by the computing system, sorting and prioritizing is based on interaction between the patient and the computing system as well as one or more changes in the sensor environment reported to the computing system by the electronic signal from the sensor.
